(19) United States
(12) Patent Application Publication
(10) Pub. No.: US 2002/0102536 A1
(43) Pub. Date: Aug. 1, 2002

(54) Preparation of human or animal herpesviruses

(75) Inventor: Michael E.G. Boursnell, Cambridge (GB)

Correspondence Address: KLARQUIST SPARKMAN, LLP, 121 SW SALMON STREET, SUITE 1600, PORTLAND, OR, 97204, US

(73) Assignee: Xenova Research Limited

(21) Appl. No.: 09/990,141

(22) Filed: Nov. 20, 2001

Related U.S. Application Data
(60) Provisional application No. 60/252,665, filed on Nov. 20, 2000.

(30) Foreign Application Priority Data
Nov. 20, 2000 (GB) PCT GB00/04415
Nov. 19, 1999 (GB) 9927353.4

(51) Int. Cl. C12Q 1/70; A61K 39/245; A61K 39/255; A61K 39/265; A61K 39/27; C12N 7/00; C12N 7/01; C12N 7/02; C12N 5/00; C12N 5/02

(52) U.S. Cl. 435/5; 435/235.1; 435/325; 435/239; 424/229.1

(57) ABSTRACT

Culture of human oranimal herpesviruses, e.g. disabled mutant herpesviruses, can be carried out on primary cells which have been made recombinant so as to express a first gene that extends their culturable life and a second gene derived from the virus. The virus products can be used in vaccines or gene delivery to cells.

### FIELD OF THE INVENTION

This invention relates to the field of viruses and mutant viruses, which can be used for or in vaccines or for gene delivery to cells, among other purposes. The invention also relates to methods for their production and manufacture, including cell cultures adapted to that purpose.

### BACKGROUND OF THE INVENTION

Production of virus mutants requires a host cell culture capable of supporting propagation of the virus to be produced, and it is known to produce viruses for vaccine and other purposes using any of a variety of cell lines. Many of these, e.g. Vero cells, are immortal or immortalised cell lines, having a transformed phenotype.

Among known culturable viruses for vaccine use are those described in WO 92/05263 (Immunology Ltd: Inglis et al) and WO 94/21807 (Cantab Pharma Res: Inglis et al), and in C S Forrester et al, (1992) J Virol 66: pp 341 et seq., which lack a gene essential for the production of infectious new virus particles from a cell infected by the mutant virus, and accordingly require to be cultured on cell which have been made recombinant so as to be able to express a heterologous gene, e.g. a herpesviral gH gene, having the corresponding viral gene function. Further examples are given in WO 96/26267 (Cantab Pharmaceuticals. Inglis et al) The cited publications also give examples of methods for making such recombinant cells and deletant viruses. It has also been proposed to provide transgenic animals which carry such a viral gene (WO 95/03399, Cantab Pharmaceuticals: Inglis et al), to act as a source of suitable cells

It remains desirable, however, to provide improved methods and materials for facilitating the culture of viruses, including defective mutant viruses, on cells that are not immortalised cell lines and do not have a transformed phenotype.

### SUMMARY OF THE INVENTION

The present invention provides methods of culturing human or animal herpesviruses, particularly for example mutant herpesviruses such as mutant cytomegaloviruses, especially under conditions in which the cultured virus products are usable for pharmaceutical purposes. Such processes can comprise: (i) providing a cell culture of mammalian primary cells, said cells having been made recombinant in respect of a first gene that can be expressed in said cells, said first gene encoding a gene product which has a function that extends the culturable life of said primary cells, but does not confer on said cells a transformed cell phenotype, wherein said cells also have been made recombinant and able to express a heterologous second gene of which the gene product is desired in connection with the propagation of said mutant virus, followed by (ii) preparing a cell bank of said cell culture, (iii) making a production cell culture from said cell bank, (iv) infecting said production cell culture with a mutant herpesvirus to be cultured, and (v) harvesting progeny virus from said production culture. The process is preferably carried out using primary cells which have not yet undergone more than about 30 population doublings, preferably about 25 doublings or less, e.g. since their isolation from their human or animal tissue source, e.g. to the start of the process, although in some cases it may be necessary to arrive at the cell banking stage when the cells have undergone more population doublings than this, e.g. up to 40 or 45 population doublings. The progeny viruses can if desired be formulated for pharmaceutical use.

In certain embodiments, according to the present invention, there is provided a method of culturing human or animal mutant herpesviruses, particularly for example cytomegaloviruses, especially for example under conditions in which the cultured virus products are usable for pharmaceutical purposes, which comprises providing a cell culture of human or animal primary cells which can stably express a gene product with the function of human or animal telomerase reverse transcriptase (hTERT) and can also express at least one viral gene heterologous to the human or animal cells, for example a viral gH gene, needed for the cell line to support propagation of a mutant virus, preparing a cell bank of said cell culture, from said cell bank making a production cell culture, infecting said production culture with the mutant viruses to be cultured, isolating progeny mutant viruses from said production culture, and formulating said progeny viruses for pharmaceutical use.

The invention can facilitate in particular the culture of mutant viruses for pharmaceutical use when for some reason it may not be desirable or practically useful to culture the viruses on cells with an immortalised (transformed) phenotype. This can be e.g. because of actual or suspected oncological safety risks in respect of the use of culture products from transformed cell lines. Where virus mutants are to be cultured on cell lines for which molecular genetic manipulations are needed in order to provide the cell line with an expressible heterologous gene in order that the cell line can support propagation of a desired mutant virus, it can happen that such manipulations when carried out on non-immortalised cell lines may involve culture processes which are so extensive that they leave too short a culturable life span in the resulting recombinant cells for sufficient economic virus production to be achieved for use before the cells die.

According to certain embodiments of the invention, cell cultures used to make the desired viruses are made (a) recombinant in respect of the wanted heterologous gene for support of the virus, and (b) also of extended culturable life span, by virtue of their incorporation of the hTERT gene function, without their being immortalised or acquiring transformed phenotype. Particularly suitable for such treatment are cells originating in lung fibroblasts, such as human foetal lung fibroblast cells well-known and available as MRC-5 cells, or originating in other fibroblasts such as human foreskin fibroblasts.

It is conventional in the art to prepare cell banks of cultured mammalian cells: this can involve growing a quantity of the cell material to be banked, under ordinary culture conditions, often in quantities that require a number of culture vessels; trypsinizing the cells of the resulting cultures to allow their suspension; pooling the suspended cells to make a generally uniform suspension of cells from the culture; gently mixing with a cryoprotectant (for example 10% dimethylsuphoxide 90% foetal calf serum); sealing aliquots of the cell suspension in ampoules; and freezing the aliquots, e.g. in liquid nitrogen. Aliquots of the homogeneous banked cells can then be taken for any desired purpose such as production culture and/or characterization for purity, viability, etc. Each aliquot ampoule can conveniently be arranged to contain of the order of 10ˆ 6 to 10ˆ 7 cells. Production cultures are usually made by unsealing an ampoule of the cell bank, thawing the contents, and transfer into ordinary cell culture medium. These or equivalent steps can form part of processes according to the invention.

According to examples of the invention, the viruses can be human or non-human animal (mammalian) herpesviruses, e.g. human herpes simplex virus or human cytomegalovirus, e.g. those which have been made deletant in respect of an essential gene such as the viral glycoprotein gH or gL gene, e.g. for use as vaccines and/or as gene delivery vectors. Genetically disabled virus vaccines and vectors are described in WO 92/05263 (Immunology Ltd: Inglis et al) and WO 94/21807 and WO 96/26267 (Cantab Pharmaceuticals: Inglis et al).

Cells on which such viruses can be cultured include for example human MRC-5 cells which have been made recombinant for hTERT and for a corresponding essential viral gene such as the gH gene, or on other cell types which have been made recombinant for hTERT and for the corresponding viral gene. Further examples of primary cells which can be made of extended life in a similar way include human CD8+ T-cells (described in E Hooijberg et al, J Immunol 165(8) (2000) pp 4239-4245), and human retinal pigment epithelial cells and foreskin fibroblasts (described in AG Bodnar et al, Science (1998) 279 (5349) pp 349-352).

In modifications of the invention, the process can also be used to culture e.g. attenuated viruses which do not require a viral gene to be introduced into the host cell.

Particular embodiments of the invention can for example be carried out as follows and are further described in connection with the accompanying drawings, in which:

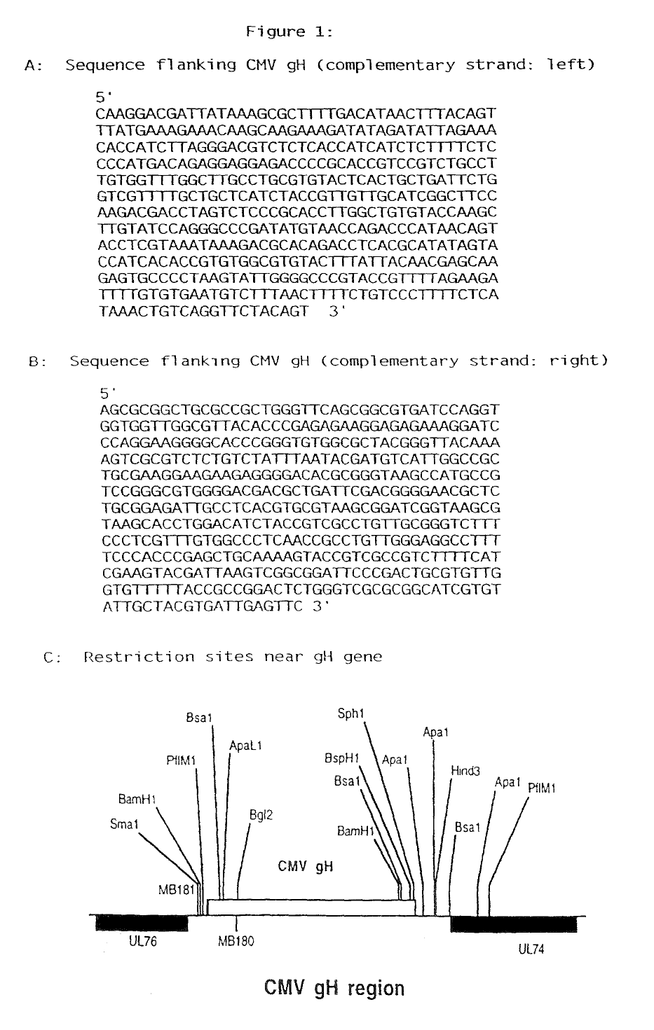

FIG. 1 shows (1A and 1B) nucleic acid sequences flanking the complementary strand of a CMV gH gene, to left and right respectively, and further shows (1C) neighbouring restriction sites: these sequences can be used in the construction of a mutant virus for culture as described herein, and the restriction sites can be used to excise a DNA sequence encoding the gH gene for insertion via a cloning vector and under control of a promoter into primary cells as described herein.

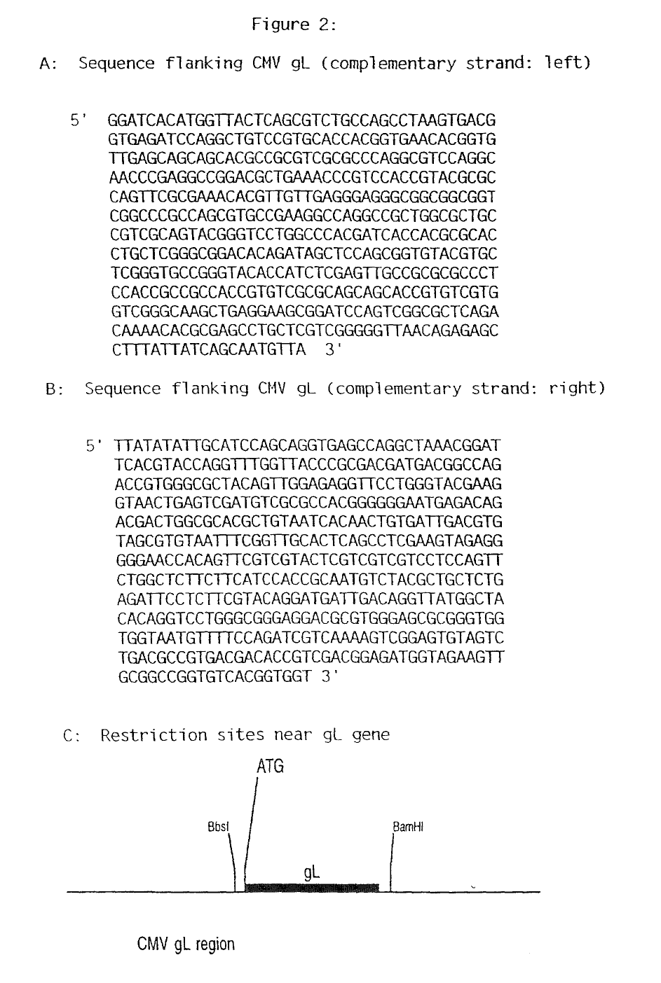

FIG. 2 shows (2A and 2B) nucleic acid sequences flanking the complementary strand of a CMV gL gene, to left and right respectively, and further shows (2C) neighbouring restriction sites: these sequences can be used in the construction of a mutant virus for culture as described herein, and the restriction sites can be used to excise a DNA sequence encoding the gL gene for insertion via a cloning vector and under control of a promoter into primary cells as described herein.

In carrying out the present invention, the person skilled in the art can make use of per-se well-known techniques of molecular biology and virology including recombinant DNA techniques and readily performable adaptations thereof, including techniques described in the following generally-known works of reference: Sambrook et al, ‘Molecular Cloning: A Laboratory Manual’ (2nd Edition, 1989) (and its predecessor edition by Maniatis et al, ‘Molecular Cloning: A Laboratory Manual’, 1982); ‘DNA Cloning: A Practical Approach’ (ed. D Glover); ‘Oligonucleotide Synthesis’ (ed. N Gait); ‘Nucleic Acid Hybridization’ (eds. B Hames et al 1985); ‘Transcription and Translation’ (eds. B Hames et al, 1984); ‘Animal Cell Culture’ (ed. R Freshney, 1986); and ‘A Practical Guide to Molecular Cloning’ (Perbal, 1984), among others.

All documents including patents and patent applications cited herein are hereby incorporated by reference in their entirety.

Examples of the invention will be described with respect to human cytomegalovirus

Human cytomegalovirus (CMV) is known as a betaherpesvirus which can cause clinically serious disease, especially in immunocompromised subjects. A complete human-cytomegalovirus genome sequence is known and is reviewed in MS Chee et al, “Analysis of the protein-coding content of the sequence of human cytomegalovirus strain AD169” (Curr. Top. Microbiol. Immunol. 154 (1990) pp 125-170). It is known to culture CMV and to use recombinant DNA techniques to manipulate the virus and its genome. For example, U.S. Pat. No. 5,474,914 (Chiron: R Spaete), while concerned primarily with production of CMV glycoprotein gH, especially in truncated secretable form, also describes the preparation of nucleic acids and vectors encoding the CMV gH and gL glycoproteins U.S. Pat. No. 5,908,780 (American Cyanamid: T R Jones) discloses examples of CMV genes which are not essential genes but which (when expressed in CMV-infected host cells) can cause down-regulation of cell surface expression of MHC class I; and also discloses CMV mutants deleted in respect of those MHC-downregulating genes—proposing them for use as vaccine U.S. Pat. No. 5,273,876 (Syntro Corp: L J Hock et al) discloses mutation of human cytomegalovirus to form recombinant virus containing a foreign gene. Constructs of these publications can readily be adapted to substitute and include nucleic acid sequences of relevance to the present invention as pointed out herein

### EXAMPLE 1

Cell production

A culture of complementing cells expressing CMV gH or gL can be made as follows: First MRC-5 lung fibroblast cells can be made recombinant so as to express the human hTERT gene. Details for carrying out this part of the recombination can be as in K L Mackenzie et al, Exp Cell Res 259 (2000) pp 336-350, esepcially pp 337-8, or as in application WO 98/14592 (Geron Corp: T R Cech et al). The resulting hTERT expressing MRC-5 cells can be subjected to further recombination as follows.

Cut fragments containing a CMV gH sequence or gL sequence can be prepared from a preparation of CMV DNA, e.g. by the use of restriction enzymes as indicated in FIG. 1C or FIG. 2C respectively, and isolated on agarose gel. After repair of the ends and blunt-end ligation the respective construct can be cloned in an [Invitrogen] plasmid pRcCMV or pRcRSV or pZeoSV2, where it is positioned under control of a CMV IE or RSV or SV40 promoter respectively. the hTERT-expressing MRC-5 cells can then be transfected with the resulting plasmid and transfected cells selected in the usual way for neomycin or zeocin resistance respectively according to the chosen piasmid. The result is to introduce the gH or gL gene and enable its expression in the hTERT expressing MRC-5 cells. Alternative promoters include CMV (e.g. virus-inducible) promoters that can be incorporated into one of the abovementioned vectors in place of the promoter supplied in the vector.

Selected cells showing appropriate antibiotic resistance can be tested for production of CMV gH or gL protein by use of anti-CMV antibody (preferably specific anti-gH or -gL antibody), and selected cells testing positive can be retained for production of mutant CMV viruses. These cells are referred to as the complementing cells for mutant virus culture, and the parent cells can be used as described below as control cells for culture of the wild-type virus.

Virus production

DNA can be prepared having the sequence of flanking sequences surrounding CMV gH or CMV gL The sequences 500 bp to the left and to the right of each of the gH and gL DNA sequences are given in FIGS. 1A-1B (for gH), and 2A-2B (for gL) respectively. (These are complementary sequences, and relate to the corresponding opposite flanks of the coding sequences.) For the gH case, these can be made by using PCR with CMV genomic DNA and oligonucleotide primers with sequence taken from the left flank beginning and end, and from the right flank beginning and end, of the gH flanking sequences—and correspondingly for the gL case.

These sequences can be joined (in order 5′-3′ from left flank to right flank as shown) with (between them) a reporter gene such as a GFP gene sequence under the control of a suitable promoter such as CMV IE promoter or (preferably) an SV40 or RSV promoter, to form a GFP-encoding construct to be used in a first virus recombination step.

Other portions of the flanking sequences can be joined directly to form a second construct to be used in a second virus recombination step.

After culture in a chosen cloning vector, the GFP-flanking sequences can be used for homologous recombination with CMV virus in a first recombination step with selection for fluorescent plaques showing growth on the complementing cells. Those clones are selected which also show no growth on the control cells.

A second recombination can be carried out with the second construct of flanking sequences described above, this time with selection for non-fluorescent plaques (white plaques) on the complemeting cell line. Those clones are selected which show white non-fluorescent plaques on the complementing cells and also show no growth on the control cells.

The viruses resulting from the examples particularly described above are gH-(or gL-) defective mutant CMV which can be grown on the complementing cell lines as described but not on control cell lines, and these can be produced and prepared as a pharmaceutical immunogen for vaccine use by usual purification and sterile formulation steps wityh suitable excipients to give doses in for example the range 10\hat{4}-10\hat{8}pfu virus per dose.

The example described in relation to a gH- mutant can be adapted in order to form a gL- mutant by carrying out the example in generally similar manner but by the use of the sequences flanking gL instead of those flanking gH.

Alternative mutant CMV viruses can be constructed by an adaptation of this example, in which a desired heterogene DNA sequence under control of a suitable promoter is inserted midway between the joined left and right flanking sequences as specified above for gH or for gL as may be desired. Altenatively a restriction site (such as a pad site) which is not otherwise present in the viral genome can be inserted instead of a heterogene, so that a heterogene can be inserted later by using a corresponding restriction enzyme on the viral DNA.

Without limitation, other examples of the invention can also be carried out by using a culture of MRC-5 cells that have been transformed or transfected with hTERT, and additionally transformed/transfected with the gH (and/or gL) gene from hCMV. Such cells can support the growth of gH-(and/or gL-) gene-defective hCMV virus, e.g. hCMV virus that has been entirely deleted in respect of its native gH and in certain examples made to carry a heterologous gene, e.g. at the site of deletion of the essential gH or gL gene, said heterogene encoding an antigen or an immunomodulatory protein such as a cytokine.

Several alternative examples can be arranged in order to make such cells transformed or transfected with hTERT: polynucleotide sequences corresponding to hTERT (human telomerase reverse transcriptase protein) can be provided for example by reference to sequences ID 113 and 117 (and FIG. 53) in specification WO 98/14592 m (Geron Corp et al: T R Cech et al), in which description at pages 5, 12 and 146 is also relevant. A plasmid vector containing the hTERT polynucleotide sequence under control of a suitable promoter can thus be prepared and introduced into MRC-5 cells (or in other examples, other suitable cells) by adaptation of known or standard methods. Preferably, primary cells such as MRC-5 cells that have undergone fewer than about 10 population doublings since isolation can be used for this purpose.

The hCMV gH gene is further described, with its sequence, in M P Cranage et al, J Viol (1988) 62(4) pp 1416-1422 (at FIG. 2 and page 1418), and its cloning is described in the same article from page 1416, col.2 to page 1417 col.1. A plasmid vector containing the CMV gH gene can be constructed by adaptation of standard methods and inserted into for example MRC-5 cells (that have also been transfected/transformed with the hTERT gene) using per-se known standard methods readily adaptable to genes of interest in the present context.

The CMV glycoprotein L gene has been further described (in the case of the guinea-pig CMV) as the product of gene UL115 (J C Paglino et al, Arch Virol 1999, vol 144(3), pp 447-462). Accordingly, a corresponding hCMV gL gene construct can for example be isolated by adaptation of standard methods, from an Xbal E fragment of hCMV Towne, which contains the UL119-UL115 transcription unit, the arrangement of which is described in M P Leatham et al, J Virol (1991) 65(11) pp 6144-6153, particularly in FIG. 10, page 6151. The CMV gL aminoacid sequence is available in the same publication (at page 6150, FIG. 9(c)) and a gL gene preparation can alternatively be obtained by adaptation of standard methods using a mixture of degenerate primers to match the 5′ and 3′ ends of the aminoacid sequence corresponding to UL115.

The introduction of the gH gene and/or gL gene into the MRC-5 cells can be effected by methods analogous to those set out in the references cited herein.

Deletion of gH and/or gL genes from hCMV can be achieved by adaptation of the methods described in references cited hereinabove.

Complementing cell lines such those prepared as indicated above can then be used to culture hCMV mutant viruses that are deletant in respect of a corresponding gene or genes, especially for example gH and/or gL genes, e.g. by use or adaptation of known culture methods for human cytomegalovirus on MRC-5 cells, as described for example in M P Cranage et al, EMBO J, (1986) 5:3057-3063 (see esp. page 3062 col 2).

Further examples of CMV sequences useful in the context of further embodiments of the present invention are present for example in the complete genome sequence of human cytomegalovirus (human herpesvirus 5), strain AD169, submitted Dec. 6, 1989 to the Genbank database where it is available at accession reference X17403 (NID g59591) (version X17403.1 GI:59591), bases 1 to 229354, (M Chee et al).

Further information about CMV sequences is available in M S Chee et al (Curr. Top. Microbiol. Immunol. 154, 125-169 (1990)); and in A T Bankier et al, “The DNA sequence of the human cytomegalovirus genome”, DNA Seq. 2 (1), 1-12 (1991).

Alternative cell substrates for culture of CMV include primary neuronal cells (I Kosugi et al, Acta Neuropathol 1998 96: 239-247); and primary retinal pigment epithelial cells (RPE) (B Bodaghi et al, J Immunol 1999 162(2): 957-964).

Non-human animal mutant cytomegaloviruses are also within the scope of the present invention and their culture and preparation and use provide further examples thereof, e.g. for their use as model agents and vaccines for example for study of the pathology of the human cytomegalovirus: for example a defective guinea-pig CMV (GPCMV) can be made by deletion of the GPCMV gL gene from GPCMV and culture on suitable cells made recombinant with the corresponding GPCMV gene. The GPCMV gene is described and available on the basis of J C Paglino et al, Arch Virol 1999 144(3) p 447-462.

Mutant viruses can be made according to the present invention so as to carry genes as vectors, e.g. in any of the ways indicated in specification WO 96/26267 (Cantab Pharmaceuticals. S C Inglis et al), and can be used to deliver genes to target cells either by in-vivo or ex-vivo methods of administering the virus to the cell so that the gene can be expressed in the cell, also contemplated as part of the present invention.

Thus culture of human or animal herpesviruses, e.g. disabled mutant herpesviruses, can be carried out on primary cells which have been made recombinant so as to express a first gene that extends their culturable life and a chosen second gene, e.g. one derived from the virus to be cultured. The virus products can be used in vaccines or gene delivery to cells.

The present invention further extends to recombinant viruses and recombinant cells and preparations thereof, and methods for their construction and their production, on the basis of the present disclosure. The invention is susceptible to modifications and variations readily performable by those skilled in the art: the present disclosure extends to combinations and subcombinations of the features indicated or descibed herein or in the appended claims The publications mentioned herein are incorporated by reference in their entirety for all purposes

1. A method of culturing a mutant herpesvirus, which comprises: (i) providing a cell culture of mammalian primary cells, said cells having been made recombinant in respect of a first gene that can be expressed in said cells, said first gene encoding a gene product which has a function that extends the culturable life of said primary cells, but does not confer on said cells a transformed cell phenotype, wherein said cells also have been made recombinant and able to express a heterologous second gene of which the gene product is desired in connection with the propagation of said mutant virus, followed by
(ii) preparing a cell bank of said cell culture,
(iii) making a production cell culture from said cell bank,
(iv) infecting said production cell culture with a mutant herpesvirus to be cultured, and
(v) harvesting progeny virus from said production culture.

2. A method according to claim 1, wherein the gene product of said first gene has the function of telomerase reverse transcriptase.

3. A method according to claim 1, wherein the mutant herpesvirus is a herpes simplex virus or a human cytomegalovirus.

4. A method according to claim 1, wherein the heterologous second gene is a viral gene needed for the cell line to support propagation of said mutant virus.

5. A method according to claim 4, wherein the second gene is a viral gH gene or gL gene.

6. A method according to claim 1, wherein the primary cells are selected from fibroblast cells such as MRC-5 cells. primary retinal pigment epithelial cells, and primary neuronal cells.

7. A method according to any of claims 1 to 6, wherein said primary cells have undergone not more than about 30 population doublings in culture previous to the introduction of said first or second gene into said cells.

8. A mutant virus producible by a method according to any one of claims 1-7.

9. A sterile pharmaceutical formulation comprising a mutant virus according to claim 8, together with a pharmaceutically acceptable excipient.

10. Use of a mutant virus according to claim 8, in the manufacture of a composition to deliver genes to target cells ex vivo or for administration to a subject to deliver genes to target cells of said subject, or to stimulate an immune response in said subject.

11. Use of a cell culture as defined in claim 1 for the manufacture of herpesvirus.

12. Use of a cell culture as defined in claim 1, wherein cells of said culture are derived from fibroblast cells and have been made recombinant so as to express hTERT gene and an essential gene of human cytomegalovirus, for the production of human cytomegaloviruses.
